(19) United States
(12) Patent Application Publication
(10) Pub. No.: US 2015/0272999 A1
(43) Pub. Date: Oct. 1, 2015

(54) RETROGRADE CORONARY VEIN DELIVERY OF STEM CELLS

(71) Applicant: Banner Health, Phoenix, AZ (US)

(72) Inventor: Mohamed A Gaballa, Peoria, AZ (US)

(21) Appl. No.: 14/673,873

(22) Filed: Mar. 31, 2015

Related U.S. Application Data
(60) Provisional application No. 61/973,253, filed on Mar. 31, 2014.

(51) Int. Cl. A61K 35/34

(52) U.S. Cl. CPC A61K 35/34

(57) ABSTRACT

The present invention relates to the delivery of stem cells to heart tissue by retrograde coronary vein infusion. The invention also provides methods useful for treating subjects with heart disease.

### RELATED APPLICATIONS

This application claims priority to U.S. Provisional Patent Application Ser. No. 61/973,253, filed on Mar. 31, 2014, which is hereby incorporated by reference herein in its entirety.

### STATEMENT OF RIGHTS UNDER FEDERALLY-SPONSORED RESEARCH

This invention was made with government support under NIH RO1 AG027263, awarded by the National Institute of Aging. The government has certain rights in this invention.

### FIELD OF THE INVENTION

The present invention relates to the delivery of stem cells to heart tissue by retrograde coronary vein infusion. The invention also provides methods useful for treating subjects with heart disease.

### BACKGROUND OF THE INVENTION

Stem cells derived from a human subject are potentially useful for a variety purposes, including regeneration of damaged tissues, reproduction, and as cellular models that could inform personal medicine, including diagnoses, treatments to alleviate a condition of disease or disorder, or warnings of adverse reaction to a potential treatment. Stem cells isolated from cardiac explant-derived cells have been shown to improve cardiac function after myocardial infarction (MI). Current obstacles to clinical use of stem cells for the treatment of heart disease include inefficient delivery and implantation of exogenous stem cells in heart tissue. To fully realize the therapeutic potential of these cells, it is essential to develop a safe and efficient delivery method.

Thus, there is a need in the art for methods for the delivery of autologous pluripotent stem cells to a human subject. The present invention meets this need by providing a safe and efficient delivery method for stem cell therapy. The present invention further provides methods useful for treating heart disease.

### SUMMARY OF THE INVENTION

The present invention provides methods for retrograde coronary vein delivery of stem cells to a subject's heart. The invention also provides methods useful for treating subjects with heart disease.

In some embodiments, the invention provides methods of retrograde coronary vein delivery of stem cells to a subject's heart comprising the steps of: inserting a catheter into a right atrium of the heart; occluding one or more blood vessels of the heart; and infusing through the catheter a solution comprising stem cells to the atrium of the subject's heart, thereby delivering stem cells to the subject's heart. In some embodiments, the one or more blood vessels is selected from the group consisting of inferior vena cava, superior vena cava, and pulmonary artery. In other embodiments, the solution is infused for approximately 5 seconds, approximately 10 seconds, approximately 20 seconds, approximately 30 seconds, approximately 40 seconds, approximately 50 seconds, approximate 1 minute, approximately 2 minutes, approximately 5 minutes, approximately 10 minutes, approximately 20 minutes, or approximately 30 minutes. In yet other embodiments, the volume of the solution is approximately 100 ul, 200 ul, 300 ul, 400 ul, 500 ul, 1 ml, 2 ml, 3 ml, 4 ml, 5 ml, 10 ml, 20 ml, 30 ml, 40 ml, or 50 ml. In still other embodiments, the number of stem cells in the solution comprises approximately 100 thousand, 250 thousand, 500 thousand, 1 million, 2 million, 3 million, 4 million, 5 million, 10 million, 20 million, or 50 million. In some embodiments, the stem cells express c-Kit. In certain aspects, the stem cells do not express CD45 or CD34. In other aspects, the stem cells further express Nanog, Flk-1/KDR, and Ki67. In still other aspects, the stem cells further express one or more markers comprising Nanog, Sox1, Oct3/4, Isl1, Nkx2.5, GATA4. In other embodiments, the solution further comprises serum-free DMEM medium. In yet other embodiments, the methods further comprise treating the stem cells with one or more of the agents selected from the group consisting of TGF-beta, mocetinostat, and VEGF. In still other embodiments, the stem cells are progenitor cells isolated from cardiac explant-derived cells.

In other embodiments, the present invention provides methods for treating a subject having or suspected of having a heart disease, the method comprising, inserting a catheter into a right atrium of the heart; occluding one or more blood vessels of the heart; and infusing through the catheter a solution comprising stem cells to the atrium of the subject's heart, thereby treating the subject. In other embodiments, the subject has a heart disease selected from the group consisting of chronic heart failure, myocardial infarction, congestive heart failure, congenital heart disease, cardiomyopathy, pericarditis, angina, and coronary artery disease.

These and other embodiments of the present invention will readily occur to those of skill in the art in light of the disclosure herein, and all such embodiments are specifically contemplated.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items. It must be noted that as used herein and in the appended claims, the singular forms “a,” “an,” and “the” include plural references unless context clearly dictates otherwise.

### BRIEF DESCRIPTION OF THE DRAWINGS

FIGS. 1A, 1B, 1C, 1D, 1E, and 1F set forth data showing characterization data for explant-derived stem cells.

FIG. 2 sets forth an illustration of the RCV infusion procedure according to an embodiment of the present invention.

FIGS. 3A, 3B, 3C, 3D, and 3E set forth data showing retention and distribution of transplanted stem cells.

FIGS. 4A, 4B, and 4C set forth data showing RCV infused stem cells contributed to cardiac lineage cells in vivo.

FIGS. 5A, 5B, 5C, 5D, and 5E set forth data showing RCV infused stem cells effects on cardiac remodeling following myocardial infarction (MI).

FIGS. 6A, 6B, 6C, and 6D set forth data showing RCV infused stem cells effect on immune response in vivo.

### DESCRIPTION OF THE INVENTION

It is to be understood that the invention is not limited to the particular methodologies, protocols, cell lines, assays, and reagents described herein, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a fragment” includes a plurality of such fragments, a reference to an “antibody” is a reference to one or more antibodies and to equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C.C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

The present invention relates, in part, to the discovery that retrograde coronary vein (RCV) delivery of stem cells enhances their distribution and transplantation into heart tissue. The present invention provides methods for delivery of stem cells to heart tissue by retrograde coronary vein infusion. The invention also provides methods useful for treating subjects with heart disease.

The section headings are used herein for organizational purposes only, and are not to be construed as in any way limiting the subject matter described herein.

### Stem Cells

Stem cells are undifferentiated cells defined by their ability at the single cell level to both self-renew and differentiate to produce progeny cells, including self-renewing progenitors, non-renewing progenitors, and terminally differentiated cells. Stem cells, depending on their level of differentiation, are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (1) totipotent, which is able to give rise to all embryonic and extraembryonic cell types; (2) pluripotent, which is able to give rise to all embryonic cell types. i.e., endoderm, mesoderm, and ectoderm; (3) multipotent, which is able to give rise to a subset of cell lineages, but all within a particular tissue, organ, or physiological system (for example, hematopoietic stem cells (HSC) can produce progeny that include HSC (self-renewal), blood cell restricted oligopotent progenitors and the cell types and elements (e.g., platelets) that are normal components of the blood); (4) oligopotent, which is able to give rise to a more restricted subset of cell lineages than multipotent stem cells; and (5) unipotent, which is able to give rise to a single cell lineage (e.g., spermatogenic stem cells).

Stem cells useful in the compositions and methods of the present invention include embryonic cells of various types, exemplified by human embryonic stem (hES) cells, described by Thomson et al. (1998) Science 282:1145; U.S. Pat. No. 7,615,374; U.S. Pat. No. 7,611,852; U.S. Pat. No. 7,582,479; U.S. Pat. No. 7,514,260; U.S. Pat. No. 7,439,064, U.S. Pat. No. 7,390,657; U.S. Pat. No. 7,220,584; U.S. Pat. No. 7,217,569; U.S. Pat. No. 7,148,062; U.S. Pat. No. 7,029,913; U.S. Pat. No. 6,887,706; U.S. Pat. No. 6,613,568; U.S. Pat. No. 6,602,711; U.S. Pat. No. 6,280,718; U.S. Pat. No. 6,200,806; and U.S. Pat. No. 5,843,780, each of which is herein incorporated in their entirety by reference; and human embryonic germ (hEG) cells (Shambloft et al., Proc. Natl. Acad. Sci. USA 95:13726, 1998). Other useful stem cells are lineage committed stem cells, such as hematopoietic or pancreatic stem cells. Examples of multipotent cells useful in methods provided herein include, but are not limited to, human umbilical vein endothelial (HuVEC) cells, human umbilical artery smooth muscle (HuASMC) cells, human differentiated stem (HKB-II) cells, cardiac stem cells, and human mesenchymal stem (hMSC) cells.

Adult stem cells are generally limited to differentiating into different cell types of their tissue of origin. However, if the starting stem cells are derived from the inner cell mass of the embryo, they can give rise to all cell types of the body derived from the three embryonic germ layers: endoderm, mesoderm and ectoderm. Stem cells with this property are said to be pluripotent. Embryonic stem cells are one kind of pluripotent stem cell. Somatic stem cells have major advantages, for example, using somatic stem cells allows a patient's own cells to be expanded in culture and then re-introduced into the patient. Of course, induced pluripotent stem cells (iPS cells) from a patient provide a source of cells that can be expanded and re-introduced to the patient, before or after stimulation to differentiate to a desired lineage of phenotype.

Cells derived from embryonic sources can include embryonic stem cells or stem cell lines obtained from a stem cell bank or other recognized depository institution. Other means of producing stem cell lines include the method of Chung et al (2006) which comprises taking a blastomere cell from an early stage embryo prior to formation of the blastocyst (at around the 8-cell stage). The technique corresponds to the pre-implantation genetic diagnosis technique routinely practiced in assisted reproduction clinics. The single blastomere cell is then co-cultured with established ES-cell lines and then separated from them to form fully competent ES cell lines.

Embryonic stem cells are considered to be undifferentiated when they have not committed to a specific differentiation lineage. Such cells display morphological characteristics that distinguish them from differentiated cells of embryo or adult origin. Undifferentiated embryonic stem (ES) cells are easily recognized by those skilled in the art, and typically appear in the two dimensions of a microscopic view in colonies of cells with high nuclear/cytoplasmic ratios and prominent nucleoli.

In one embodiment, the stem cells are isolated prior to RCV infusion to the subject's heart. Most conventional methods to isolate a particular stem cell of interest involve positive and negative selection using markers of interest. Agents can be used to recognize stem cell markers, for instance labeled antibodies that recognize and bind to cell-surface markers or antigens on desired stem cells. Antibodies or similar agents specific for a given marker, or set of markers, can be used to separate and isolate the desired stem cells using fluorescent activated cell sorting (FACS), panning methods, magnetic particle selection, particle sorter selection and other methods known to persons skilled in the art, including density separation (Xu et al. (2002) Circ. Res. 91:501; U.S. patent application Ser. No. 20030022367) and separation based on other physical properties (Doevendans et al. (2000) J. Mol. Cell. Cardiol. 32:839-851). Alternatively, genetic selection methods can be used, where a stem cell can be genetically engineered to express a reporter protein operatively linked to a tissue-specific promoter and/or a specific gene promoter; therefore the expression of the reporter can be used for positive selection methods to isolate and enrich the desired stem cell. For example, a fluorescent reporter protein can be expressed in the desired stem cell by genetic engineering methods to operatively link the marker protein to a promoter active in a desired stem cell (Klug et al. (1996) J. Clin. Invest. 98:216-224; U.S. Pat. No. 6,737,054). Other means of positive selection include drug selection, for instance as described by Klug et al., supra, involving enrichment of desired cells by density gradient centrifugation. Negative selection can be performed, selecting and removing cells with undesired markers or characteristics, for example fibroblast markers, epithelial cell markers etc.

Undifferentiated ES cells express genes that can be used as markers to detect the presence of undifferentiated cells. The polypeptide products of such genes can be used as markers for negative selection. For example, see U.S. application Ser. No. 2003/0224411 A1; Bhattacharya (2004) Blood 103(8):2956-64; and Thomson (1998), supra., each herein incorporated by reference. Human ES cell lines express cell surface markers that characterize undifferentiated nonhuman primate ES and human EC cells, including stage-specific embryonic antigen (SSEA)-3, SSEA-4, TRA-1-60, TRA-1-81, and alkaline phosphatase. The globo-series glycolipid GL7, which carries the SSEA-4 epitope, is formed by the addition of sialic acid to the globo-series glycolipid Gb5, which carries the SSEA-3 epitope. Thus, GL7 reacts with antibodies to both SSEA-3 and SSEA-4. Undifferentiated human ES cell lines do not stain for SSEA-1, but differentiated cells stain strongly for SSEA-1. Methods for proliferating hES cells in the undifferentiated form are described in WO 99/20741, WO 01/51616, and WO 03/020920, which are herein incorporated by reference in their entirety.

In one embodiment, the methods provide for retrograde coronary vein infusion of stem cells to a subject's heart. The stem cells are selected for a characteristic of interest. In some embodiments, a wide range of markers may be used for selection. One of skill in the art will be able to select markers appropriate for the desired cell type. The characteristics of interest include expression of particular markers of interest, for example specific subpopulations of stem cells and stem cell progenitors will express specific markers. In some embodiments, stem cells used in the methods of the present invention express one or more markers selected from the group consisting of c-Kit, Nanog, Flk-1, KDR, Ki67, Sox1, Oct3/4, Nkx2.5, and GATA4.

In some embodiments, the stem cells are expanded prior to RCV infusion. The cells are optionally collected, separated, and further expanded, generating larger populations of stem cells for use in making cells of a particular cell type or cells having an enhanced efficiency of homologous recombination.

### Induced Pluripotent Stem Cells (iPS Cells)

The production of iPS cells is generally achieved by the introduction of nucleic acid sequences encoding stem cell-associated genes into an adult, somatic cell. Historically, these nucleic acids have been introduced using viral vectors and the expression of the gene products results in cells that are morphologically, biochemically, and functionally similar to pluripotent stem cells (e.g., embryonic stem cells). This process of altering a cell phenotype from a somatic cell phenotype to a pluripotent stem cell phenotype is termed reprogramming iPS cells can be generated or derived from terminally differentiated somatic cells, as well as from adult stem cells. That is, a non-pluripotent stem cell can be rendered pluripotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors. Reprogramming can be achieved by introducing a combination of stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Ten, and LIN28. In one embodiment, successful reprogramming is accomplished by introducing Oct-4, a member of the Sox family, a member of the Klf family, and a member of the Myc family to a somatic cell. In one embodiment each of Oct 4, Sox2, Nanog, c-MYC and Klf4 are used to reprogram a human stem cell.

The efficiency of reprogramming (i.e., the number of reprogrammed cells) derived from a population of starting cells can also be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135, which are incorporated herein by reference in their entirety. It is contemplated that the methods described herein can also be used in combination with a single small molecule (or a combination of small molecules) that enhances the efficiency of induced pluripotent stem cell production or that replaces one or more reprogramming factors during the reprogramming process. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), and trichostatin (TSA), among others.

To confirm the induction of pluripotent stem cells, isolated clones can be tested for the expression of a stem cell marker. Such expression identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utfl, and Nat1. Methods for detecting the expression of such markers can include, for example, RT-PCR and immunological methods that detect the presence of the encoded polypeptides. In one embodiment, detection does not involve RT-PCR, but rather focuses on detection of protein markers.

The pluripotent stem cell character of the isolated cells can be confirmed by any of a number of tests evaluating the expression of ES markers and the ability to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers further indicates that the cells are pluripotent stem cells.

### Tissue Samples

In some embodiments, stem cells used in the methods of the present invention are isolated from human tissues. Any bodily tissue may be used in the methods of the present invention, including, for example, tissue from an organ, from skin, from adipose, and from blood. Organ tissues useful for the compositions and methods of the present invention include liver, lung, heart, kidney, heart, brain, and pancreas. In certain embodiments, stem cells are isolated from heart tissue from atrial or ventricular biopsy specimens from human subjects. Such subjects may have heart disease including, for example, myocardial infarction or chronic heart failure.

### Isolation and Maintenance of Stem Cells

In some embodiments, stem cells useful in the methods of the present invention are isolated from a sample or biopsy of bodily tissue by digested by enzymatic digestion, mechanical separation, filtration, centrifugation and combinations thereof. The number and quality of the isolated stem cells can vary depending, e.g., on the quality of the tissue used, the compositions of perfusion buffer solutions, and the type and concentration of enzyme. Frequently used enzymes include, but are not limited to, collagenase, pronase, trypsin, dispase, hyaluronidase, thermolysin and pancreatin, and combinations thereof. Collagenase is most commonly used, often prepared from bacteria (e.g. from Clostridium histolyticum), and may often consist of a poorly purified blend of enzymes, which may have inconsistent enzymatic action. Some of the enzymes exhibit protease activity, which may cause unwanted reactions affecting the quality and quantity of viable/healthy cells. It is understood by those of skill in the art to use enzymes of sufficient purity and quality to obtain viable stem cell populations.

The methods of the invention comprise culturing the stem cells obtained from human tissue samples prior to RCV infusion to the subject's heart. In one embodiment, the populations of stem cells are plated onto a substrate. In the present invention, cells (e.g., stem cells) are plated onto a substrate which allows for adherence of cells thereto, i.e., a surface which is not generally repulsive to cell adhesion or attachment. This may be carried out, e.g., by plating the cells in a culture system (e.g., a culture vessel) which displays one or more substrate surfaces compatible with cell adhesion. When the said one or more substrate surfaces contact the suspension of cells (e.g., suspension in a medium) introduced into the culture system, cell adhesion between the cells and the substrate surfaces may ensue. Accordingly, the term “plating onto a substrate which allows adherence of cells thereto” refers to introducing cells into a culture system which features at least one substrate surface that is generally compatible with adherence of cells thereto, such that the plated cells can contact the said substrate surface. General principles of maintaining adherent cell cultures are well-known in the art.

As appreciated by those skilled in the art, the cells may be counted in order to facilitate subsequent plating of the cells at a desired density. Where, as in the present invention, the cells after plating may primarily adhere to a substrate surface present in the culture system (e.g., in a culture vessel), the plating density may be expressed as number of cells plated per mm² or cm² of the said substrate surface.

Typically, after plating of the stem cells of the present invention, the cell suspension is left in contact with the adherent surface to allow for adherence of cells from the cell population to the said substrate. In contacting the stem cells with adherent substrate, the cells may be advantageously suspended in an environment comprising at least a medium, in the methods of the invention typically a liquid medium, which supports the survival and/or growth of the cells. The medium may be added to the system before, together with or after the introduction of the cells thereto. The medium may be fresh, i.e., not previously used for culturing of cells, or may comprise at least a portion which has been conditioned by prior culturing of cells therein, e.g., culturing of the cells which are being plated or antecedents thereof, or culturing of cells more distantly related to or unrelated to the cells being plated.

The medium may be a suitable culture medium as described elsewhere in this specification. Preferably, the composition of the medium may have the same features, may be the same or substantially the same as the composition of medium used in the ensuing steps of culturing the attached cells. Otherwise, the medium may be different.

Cells from the stem cell population or from tissue explants of the present invention, which have adhered to the said substrate, preferably in the said environment, are subsequently cultured for at least 7 days, for at least 8 days, or for at least 9 days, for at least 10 days, at least 11, or at least 12 days, at least 13 days or at least 14 days, for at least 15 days, for at least 16 days or for at least 17 days, or even for at least 18 days, for at least 19 days or at least 21 days or more. The term “culturing” is common in the art and broadly refers to maintenance and/or growth of cells and/or progeny thereof.

In some embodiments, the stem cells may be cultured for at least between about 10 days and about 40 days, for at least between about 15 days and about 35 days, for at least between about 15 days and 21 days, such as for at least about 15, 16, 17, 18, 19 or 21 days. In some embodiments, the stem cells of the invention may be cultured for no longer than 60 days, or no longer than 50 days, or no longer than 45 days.

The tissue explants and stem cells and the further adherent stem cells are cultured in the presence of a liquid culture medium. Typically, the medium will comprise a basal medium formulation as known in the art. Many basal media formulations can be used to culture the stem cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art and it is within the skill of one in the art to modify or modulate concentrations of media and/or media supplements as necessary for the cells cultured. In some embodiments, a culture medium formulation may be explants medium (CEM) which is composed of IMDM supplemented with 10% fetal bovine serum (FBS, Lonza), 100 U/ml penicillin G, 100 μg/ml streptomycin and 2 mmol/L L-glutamine (Sigma-Aldrich). Other embodiments may employ further basal media formulations, such as chosen from the ones above.

For use in culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Also contemplated is supplementation of cell culture medium with mammalian plasma or sera. Plasma or sera often contain cellular factors and components that are necessary for viability and expansion. The use of suitable serum replacements is also contemplated (e.g., FBS).

In some embodiments, the medium comprises one or more TGF-β inhibitors, including, for example, SB431542 (Sigma-Aldrich) or SIS3 (Sigma-Aldrich).

As described, the present inventors have realized that by culturing tissue explants and stem cells for time durations as defined above, and preferably using media compositions as described above, a progenitor or stem cell of the invention emerges and proliferates. As detailed in the Examples section, the progenitor or stem cell may be distinguished from other cell types present in the primary cell culture by, among others, its expression of various markers.

The inventors also realized that the proliferation and enrichment of the culture for the said progenitor or stem cell may be further promoted by incubating an agent in the culture medium such that this enhances growth rate, differentiation potential, and pluripotency. In such conditions, the progenitor or stem cell used in the methods of the invention can advantageously proliferate and become a prevalent cell type in the cell culture. In some embodiments, the agent is TGF-β inhibitor, mocetinostat, or VEGF.

### Characterization of Stem Cells

In some embodiments, stem cells used in the methods of the present invention are identified and characterized by their expression of specific marker proteins, such as cell-surface markers. Detection and isolation of these cells can be achieved, e.g., through flow cytometry, ELISA, and/or magnetic beads. Reverse-transcription polymerase chain reaction (RT-PCR) can also be used to monitor changes in gene expression in response to differentiation. In certain embodiments, the marker proteins used to identify and characterize the stem cells are selected from the list consisting of c-Kit, Nanog, Flk-1, KDR, Ki67, Sox1, oct3/4, Isl1, Nkx2.5, and GATA4.

In some embodiments, methods of the present invention increase pluripotency markers in the isolated stem cells. Tissue explants are cultured in the presence of an effective amount of a TGF-β inhibitor, mocetinostat, or VEGF. Following incubation, the expression levels of one or more pluripotency marker is determined in the stem cells.

### Subjects

In certain embodiments of all the above-described methods, the subject is a human subject. In certain embodiments, the subject is diagnosed with or suspected of having had a disease. In other embodiments, the patient is diagnosed with or suspected of having a heart disease, or is believed to have been exposed to or to be at risk for exposure to a heart disease. In some embodiments, the subject has a heart disease selected from the group consisting of chronic heart failure, myocardial infarction, congestive heart failure, congenital heart disease, cardiomyopathy, pericarditis, angina, and coronary artery disease.

### Retrograde Coronary Vein Infusion

The present invention provides methods for the delivery of stem cells to heart tissue by retrograde coronary vein infusion. In some embodiments, the subject's right external jugular is cannulated using a catheter. The catheter is then advanced into the right atrium of the subject's heart. A number of stems cells suspended in a solution and infused for a period of time to the right atrium of the subject's heart while simultaneously and temporarily occluding one or more blood vessels of the subject's heart.

The number of stem cells infused to the subject's heart can be varied. In some embodiments, the number of stem cells in the solution comprises approximately 1, 10, 100, 10 thousand, 50 thousand, 100 thousand, 250 thousand, 500 thousand, 1 million, 2 million, 3 million, 4 million, 5 million, 10 million, 20 million, 50 million, 100 million, 500 million, or 1 billion. The volume of the solution infused into the subject's heart can be varied. In some embodiments, the volume of the solution is approximately 100 ul, 200 ul, 300 ul, 400 ul, 500 ul, 1 ml, 2 ml, 3 ml, 4 ml, 5 ml, 10 ml, 20 ml, 30 ml, 40 ml, 50 ml, 60 ml, 70 ml, 80 ml, 90 ml, 100 ml, 500 ml or 1 L. The period of time for the infusion may be varied. In some embodiments, the solution is infused for approximately 5 seconds, approximately 10 seconds, approximately 20 seconds, approximately 30 seconds, approximately 40 seconds, approximately 50 seconds, approximate 1 minute, approximately 2 minutes, approximately 5 minutes, approximately 10 minutes, approximately 20 minutes, approximately 30 minutes, or approximately 1 hour.

In general, one or more blood vessels of the heart are occluded during the infusion of the stem cells in the methods of the present invention. In a preferred embodiment, the one or more blood vessels are selected from the group consisting of inferior vena cava, superior vena cava, and pulmonary artery. In some embodiments, the solution further comprises serum-free DMEM medium.

In some embodiments, a dye may be used to confirm that the perfused solution has entered the targeted heart tissue. In one aspect of the present embodiment, 2% Evans Blue solution can be infused into the atrium to confirm that the perfused solution has entered the targeted heart tissue.

### Methods of Treatment

The present invention provides methods of treating a disease in a subject, comprising, inserting a catheter into a right atrium of the heart; occluding one or more blood vessels of the heart; and infusing through the catheter a solution comprising stem cells to the atrium of the subject's heart, thereby treating the subject. In one aspect, the human stem cells described herein can be produced from stem cells isolated from a subject having a disease. The isolated stem cells can be used to treat the disease by administering an effective amount of human stem cells to the subject. In some embodiments, an effective amount is a dosage is sufficient to generate significant numbers of new cardiomyocytes cells in the heart, and/or at least partially replace necrotic heart tissue, and/or produce a clinically significant change in heart function. A clinically significant improvement in heart performance can be determined by measuring the left ventricular ejection fraction, prior to, and after administration of cells, and determining at least a 5% increase, preferably 10% or more, in the total ejection fraction. Standard procedures are available to determine ejection fraction, as measured by blood ejected per beat. Dosages can vary from about 100-10⁷, 1000-10⁶ or 10⁴-10⁵ cells.

A wide range of diseases are recognized as being treatable with stem cell therapies. As non-limiting examples, these include disease marked by a failure of naturally occurring stem cells, such as aplastic anemia, Fanconi anemia, and paroxysmal nocturnal hemoglobinuria (PNH). Others include, for example: acute leukemias, including acute lymphoblastic leukemia (ALL), acute myelogenous leukemia (AML), acute biphenotypic leukemia and acute undifferentiated leukemia; chronic leukemias, including chronic myelogenous leukemia (CML), chronic lymphocytic leukemia (CLL), juvenile chronic myelogenous leukemia (JCML) and juvenile myelomonocytic leukemia (JMML); myeloproliferative disorders, including acute myelofibrosis, angiogenic myeloid metaplasia (myelofibrosis), polycythemia vera and essential thrombocythemia; lysosomal storage diseases, including mucopolysaccharidoses (MPS), Hurler's syndrome (MPS-IH), Scheie syndrome (MPS-IS), Hunter's syndrome (MPS-II), Sanfilippo syndrome (MPS-III), Morquio syndrome (MPS-IV), Maroteaux-Lamy Syndrome (MPS-VI), Sly syndrome, beta-glucuronidase deficiency (MPS-VII), adrenoleukodystrophy, mucolipidosis II (I-cell Disease), Krabbe disease, Gaucher's disease, Niemann-Pick disease, Wolman disease and metachromatic leukodystrophy; histiocytic disorders, including familial erythrophagocytic lymphohistiocytosis, histiocytosis-X and hemophagocytosis; phagocyte disorders, including Chediak-Higashi syndrome, chronic granulomatous disease, neutrophil actin deficiency and reticular dysgenesis; inherited platelet abnormalities, including amegakaryocytosis/congenital thrombocytopenia; plasma cell disorders, including multiple myeloma, plasma cell leukemia, and Waldenstrom's macroglobulinemia. Other malignancies treatable with stem cell therapies include but are not limited to breast cancer, Ewing sarcoma, neuroblastoma and renal cell carcinoma, among others. Also treatable with stem cell therapy are: lung disorders, including COPD and bronchial asthma; congenital immune disorders, including ataxia-telangiectasia, Kostmann syndrome, leukocyte adhesion deficiency, DiGeorge syndrome, bare lymphocyte syndrome, Omenn's syndrome, severe combined immunodeficiency (SCID), SCID with adenosine deaminase deficiency, absence of T & B cells SCID, absence of T cells, normal B cell SCID, common variable immunodeficiency and X-linked lymphoproliferative disorder; other inherited disorders, including Lesch-Nyhan syndrome, cartilage-hair hypoplasia, Glanzmann thrombasthenia, and osteopetrosis; neurological conditions, including acute and chronic stroke, traumatic brain injury, cerebral palsy, multiple sclerosis, amyotrophic lateral sclerosis and epilepsy; cardiac conditions, including atherosclerosis, congestive heart failure and myocardial infarction; metabolic disorders, including diabetes; and ocular disorders including macular degeneration and optic atrophy. Such diseases or disorders can be treated either by administration of stem cells themselves, permitting in vivo differentiation to the desired cell type with or without the administration of agents to promote the desired differentiation, or by administering stem cells differentiated to the desired cell type in vitro. Efficacy of treatment is determined by a statistically significant change in one or more indicia of the targeted disease or disorder.

Heart diseases may be treated using the methods of the invention. In particular, chronic heart failure, myocardial infarction, congestive heart failure, congenital heart disease, cardiomyopathy, pericarditis, angina, and coronary artery disease may be treated using the methods of the invention.

For compositions useful for the present methods of treatment, a therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

A therapeutically effective dose or amount of a composition used in the methods of the present invention refers to an amount or dose of the composition that results in amelioration of symptoms or a prolongation of survival in a subject. Toxicity and therapeutic efficacy of such compositions can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Compositions that exhibit high therapeutic indices are preferred.

The effective amount or therapeutically effective amount is the amount of the composition that will elicit the biological or medical response of a tissue, system, animal, or human that is being sought by the researcher, veterinarian, medical doctor, or other clinician.

These and other embodiments of the present invention will readily occur to those of ordinary skill in the art in view of the disclosure herein.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications are intended to fall within the scope of the appended claims.

### Example 1

### Retrograde Coronary Vein Infusion of Cardiac Stem Cells in Rats with Chronic Heart Failure

Cardiac stem cells (c-Kit+ cells) were transplanted via retrograde coronary vein (RCV) infusion to rats that had developed chronic heart failure (CHF) twenty-one days after myocardial infarction as follows. Two-month-old Sprague Dawley rats (Harlan Laboratories) were anesthetized and ventilated. Following a left thoracotomy, the heart was expressed, and the left anterior descending coronary artery was ligated using a 5-0 TiCron suture. The lungs were briefly hyperinflated, the chest was closed using 2-0 silk, and the rodents were allowed to recover with a pain management regiment of buprenorphine. Sham-operated animals underwent the same surgical procedure excluding left anterior descending artery occlusion. Rats were assigned randomly to four groups: 1) CHF animals RCV-infused with c-Kit+ cells (RCV/CHF), and 2) sham-operated animals RCV-infused with c-Kit+ cells (RCV/sham); and two control groups: 3) CHF animals RCV-infused with vehicle (serum-free DMEM medium); and 4) sham-operated rats RCV-infused with vehicle.

Twenty-one days after the initial MI surgery, animals were randomly assigned to cell- or vehicle-treated groups. Prior to cell delivery, scar presence was confirmed visually. The right external jugular was cannulated using a PE-50 catheter, which then advanced into the right atrium. One million GFP-labeled c-Kit+ cells were suspended in 400 μl serum-free DMEM medium and infused for 30 to 60 seconds to the right atrium, while simultaneously and temporally occluding pulmonary artery and both inferior and superior vena cava. Since all the coronary veins are occluded during cell infusion, the only exit for the cells is the cardiac great vein via coronary sinus. This same procedure was used to infuse 400 μl cell-free medium to the control groups. To confirm the targeted LV perfusion, 2% Evans Blue solution was injected using the same technique followed by heart tissue collection.

Cardiac explant outgrowth was generated as previously described (Zakharova et al. (2012) PLoS One 7:e37800). After 21 days in culture, c-Kit+ cells were separated from the cell outgrowth using magnetic beads (MACS, Miltenyi Biotec) and cultured as described in Zakharova et al. ((2012) PLoS One 7:e37800). For in vivo experiments, c-Kit+ cells were labelled with GFP lentivirus vector (Clontech). GFP expression was verified by fluorescent microscopy.

Total RNA was extracted from the c-Kit+ cells using PureLink RNA Mini Kit (Life Technologies) according to the manufacturer's protocol. RNA was then quantified with a Quanti-iT RiboGreen RNA Assay Kit and assessed using a BioTek Synergy HT Microplate Reader (excitation/emission 480/520 nm). Total RNA (200 ng) was reverse-transcribed with a QuantiTect Reverse Transcription kit (Qiagen). Real-time RT-PCR was conducted using Rower SYBR Green Master Mix (Applied Biosystems) on a StepOnePlus Real-time PCR System (Applied Biosystems). Specific primers were synthesized by Life Technologies. Data analysis was performed on StepOne software version 2.1 (Applied Biosystems) using the comparative Ct (ΔΔCt) quantitation method.

Western blotting was carried out as follows. Isolated c-Kit+ cells were lysed in RIPA buffer (Thermo Scientific) containing Halt Phosphatase and Proteinase inhibitor cocktail (Thermo Scientific) according to the manufacturer's protocol. Protein concentration was determined using a BCA Protein Assay kit (Thermo Scientific). An equal amount of protein (50 μg) was loaded in each well of 4% to 12% bis-tris gels gel (Life Sciences) and subjected to electrophoresis. Proteins were transferred to a PVDF membrane (Millipore) and then blocked with 5% nonfat dry milk in Tris-buffered saline followed by overnight incubation with primary antibodies at 4° C. Antibodies against p-Smad2/3, Smad2/3 (Cell Signaling), and Nanog (Millipore) were used. Blots were probed with an anti-β-actin (Sigma Aldrich) antibody as a loading control. Membranes were washed in Tris-buffered saline containing 0.05% Tween 20. Corresponding horseradish peroxidase-conjugated anti-rabbit or anti-mouse IgG (Invitrogen) was used as secondary antibodies Immunoreactive proteins were detected by chemiluminescence (Thermo Scientific). Band intensity was determined using FluorChem 8900 software (Alpha Innotech Corp).

Isolated c-Kit+ cells were further characterized using flow cytometry as follows. Cells were fixed in 70% ethanol and labeled with the following antibodies: c-Kit (Santa-Cruz Biotechnology), vimentin and smooth muscle actin (Abcam), and CD90 (BD Biosciences). Cells were treated with secondary antibodies corresponding to either anti-rabbit or anti-mouse IgG conjugated with Alexa 488, phycoerythrin (PE), or PE-Cy5.5 (Life Technologies). Direct labeling with FITC-conjugated CD34 and PE-Cy5.5 conjugated CD45 (BD Biosciences) antibodies was used to exclude bone marrow and hematopoietic cells. Freshly isolated bone marrow cells were used as positive controls for CD34 and CD45 labeling. For a negative control, cells were labeled with isotype IgG instead of primary antibody. Cell events were detected using a FACS Calibur flow cytometer equipped with an argon laser (BD Biosciences). Data were analyzed using CellQuest software (BD Biosciences).

Snap-frozen heart tissue was sectioned using Leica CM1900 cryostat (Leica Microsystems, Bannockburn, Ill.). For scar assessment, sections were stained with Masson's Trichrome kit (Sigma-Aldrich). In stained tissue section, scar was identified by aniline blue staining of collagen and myocardial muscle was identified by red staining Blue and red staining areas were measured using DP2-BSW (Olympus Corp) software. Scar was separated from non-fibrotic tissue or empty space based on red and blue pixel intensity thresholding. Scar percentage was calculated as a ratio of collagen enriched scar area (blue) to the whole left ventricle area (red).

C-Kit+ cell differentiation in vitro was conducted as follows. For cardiomyocyte differentiation, c-Kit+ cells were cultured in cardiac differentiation medium (EMD Millipore) supplemented with 2 μM Mocetinostat (SelleckChem) for 7 days. For endothelial differentiation, cells were treated with 10 ng/ml VEGF (R&D Systems) for 14 days. For smooth muscle differentiation, cells were treated with 10 ng/ml TGF-β (R&D Systems). After 7 days, cell differentiation was evaluated by immunostaining.

Hemodynamic statistics were collected from the rats post-MI using a pressure-volume catheter (Millar Instruments) inserted into the right carotid artery and advanced into the left ventricle. The animals were systemically anesthetized with Inactin (125 mg/kg) and intubated, and the steady-state measurements were collected prior to ventilation. The data were analyzed using PVAN 3.6. software (Millar Instruments).

The host immune reaction and inflammatory response following transplantation of stem cells according to the methods of the present invention was assessed. Inflammatory response was quantified by counting the number of neutrophils and phagocytes at 5 random areas in the infarcted zone as described previously. (Zakharova et al. (2010) Cardiovasc. Res. 87:40-9.) The inflammatory response was expressed as a number of neutrophils or phagocytes per 0.5 mm².

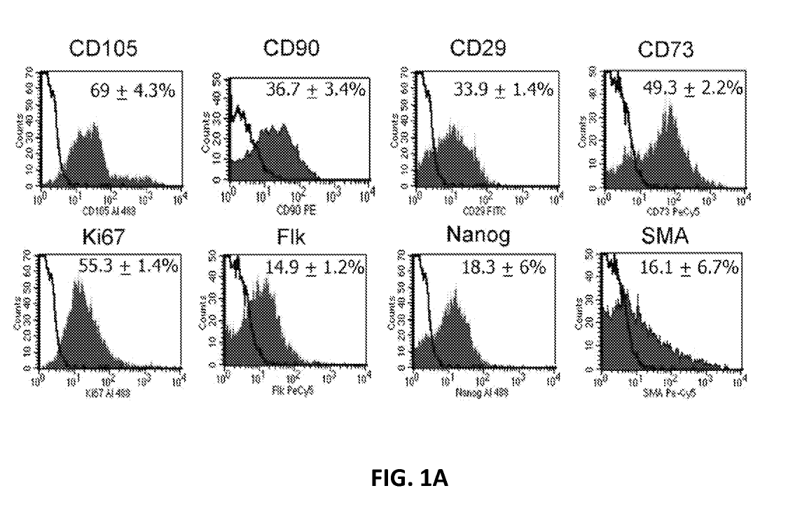

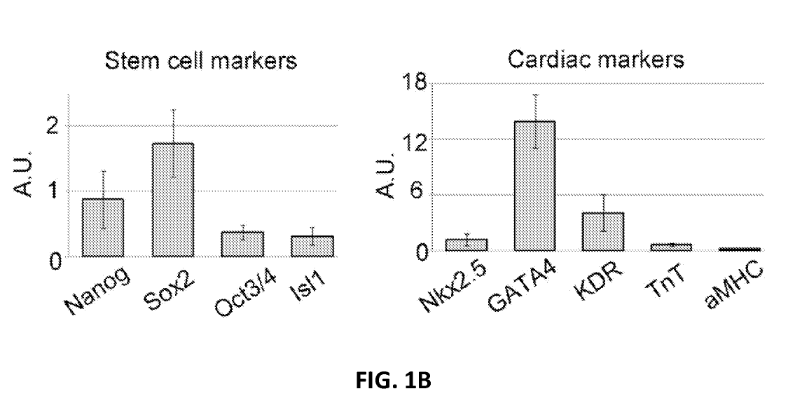

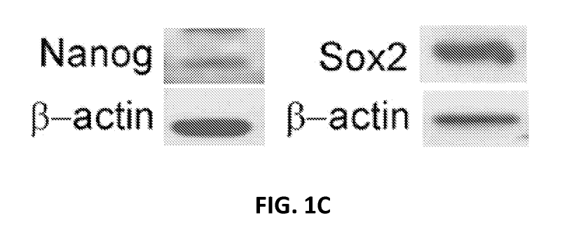

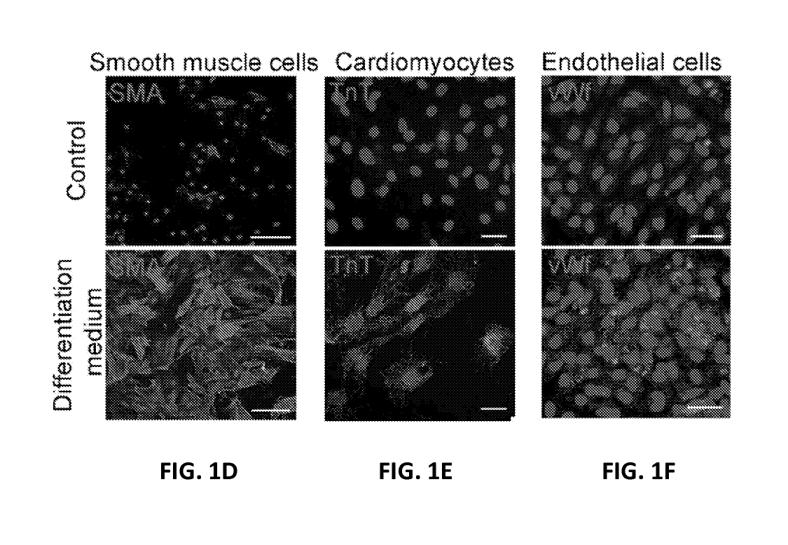

Flow cytometry analysis showed that a substantial number of c-Kit+ cells were positive for mesenchymal markers such as CD90, CD105, CD73, CD29 and SMA (FIG. 1A). In addition, c-Kit+ cells were positive for pluripotency marker, Nanog, cardiac progenitor marker, Flk-1/KDR, and proliferation marker, Ki67. (See FIG. 1A.) Real-time PCR analysis showed that c-Kit+ cells expressed pluripotency genes, Nanog and Sox2, and, at lower levels, Oct3/4 and Isl1 (FIG. 1B). Protein expressions of Nanog and Sox2 were confirmed by Western blot (FIG. 1C). C-Kit+ cells also expressed early cardiac transcription factors, Nkx2.5 and GATA4, while the expression levels of mature cardiomyocyte markers such as alpha-myosin heavy chain (αMHC) and cardiac troponin T (TnT) were low (FIG. 1B).

C-Kit+ cells were capable of differentiation into multiple cardiac lineages in vitro. Cells were cultured in various differentiation mediums and analyzed for expression of lineage-specific markers. Immunocytochemical analysis showed that c-Kit+ cells were capable to differentiate into three cardiac lineages: cardiomyocytes, smooth muscle cells, and endothelial cells. Differentiated cells were identified by labeling with TnT (cardiomyocytes), SMA (smooth muscle cells) and vWf (endothelial cells) (see FIGS. 1D-1F). Taken together, these data suggest that c-Kit+ cells maintain an immature phenotype with the ability to differentiate toward cardiac lineage cells.

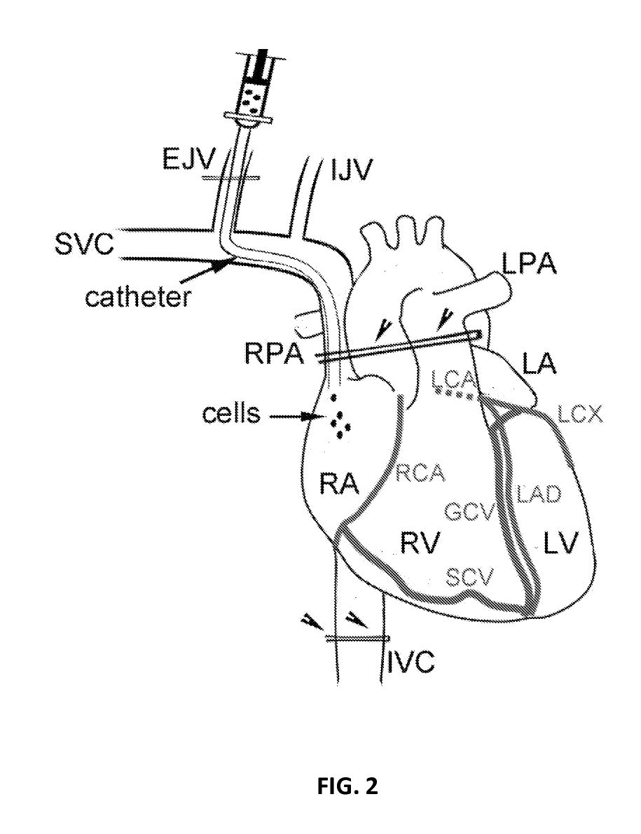

RCV infusion procedure resulted in no mortality (i.e., 100% survival rate). A schematic drawing of RCV infusion is presented in FIG. 2. Since all cardiac veins were temporarily occluded, the only exit for the cells was via the coronary sinus ostium (FIG. 2).

The effects of RCV delivered c-Kit+ cells on cardiac function were examined. At 21-days post-infusion, significant loss of function was observed in CHF group compared to sham animals. In CHF group, ejection fraction (EF) was decreased to 31.8±7.7% and left ventricle end diastolic pressure (LVEDP) was increased to 29.4±5.9 mmHg, compared to sham, confirming heart failure condition. In contrast, CHF rats transplanted with c-Kit+ cells demonstrated a significant decrease in LVEDP to 10.5±4 0 mmHg and an increase in EF to 46.8±17.5%, compared to vehicle-treated CHF. Furthermore, RCV delivery of c-Kit+ cells into sham-operated controls had no effect on LVEDP or EF. These results showed that the methods of the present invention are useful for delivering stem cells to the heart. These results further showed that the methods of the present invention are useful for treating heart disease.

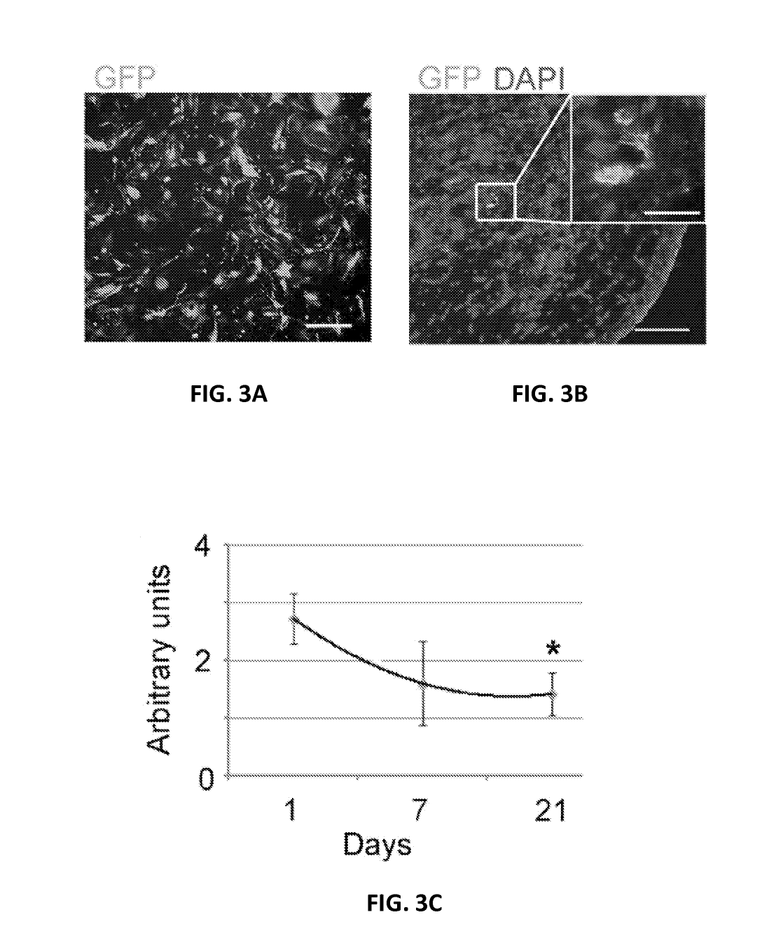

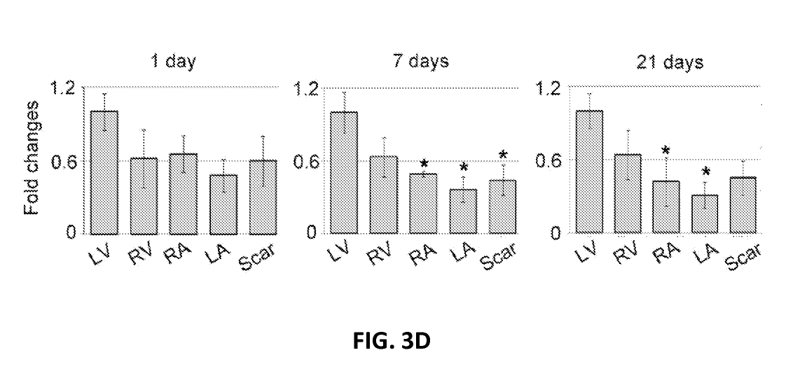

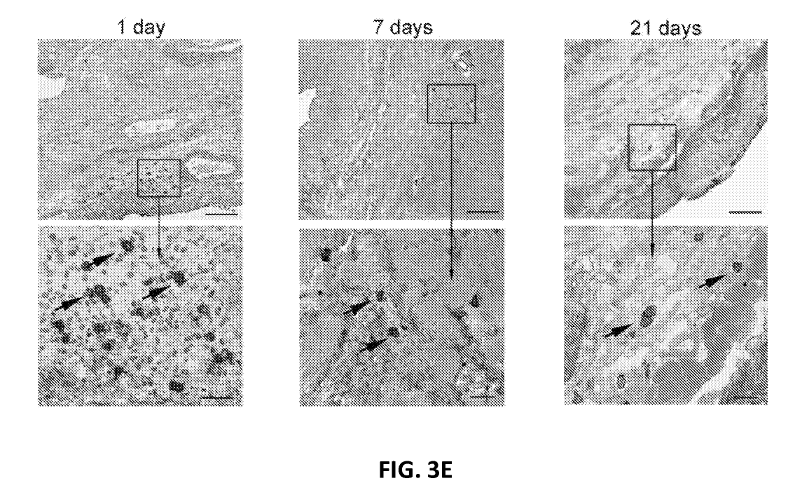

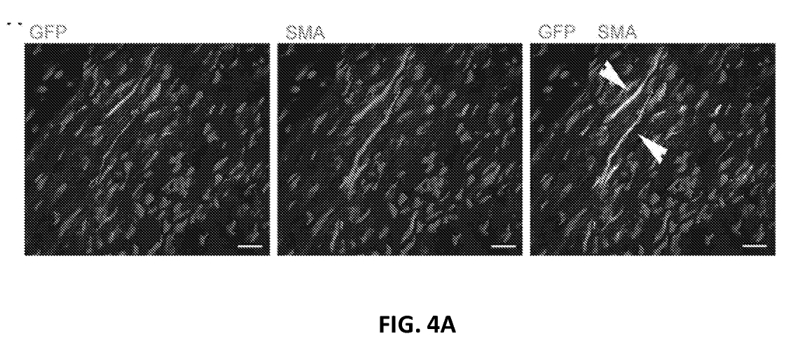

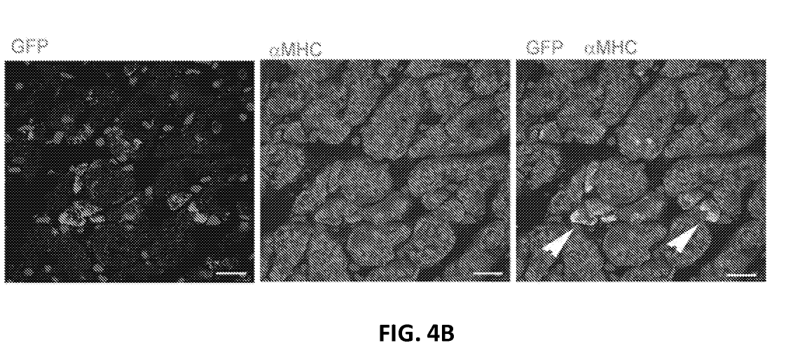

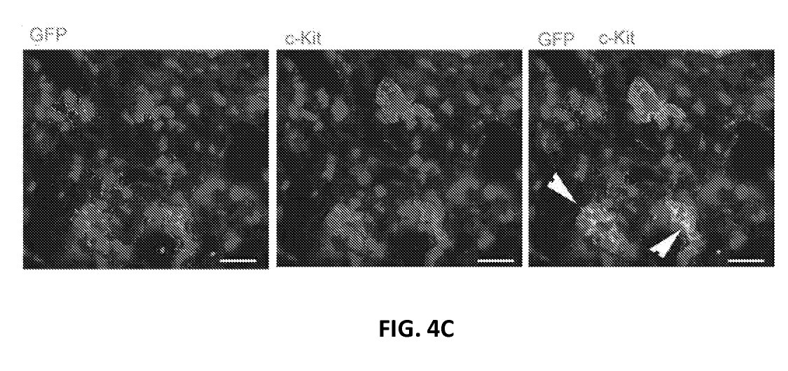

The distribution, engraftment and in vivo differentiation of RCV-transplanted c-Kit+ cells were examined To track transplanted cells in vivo, c-Kit+ cells were transduced with a GFP-carrying lentiviral vector (see FIGS. 3A and 3B). Time course of transplanted cell retention and relative cell distribution in heart compartments were estimated by measuring levels of GFP gene expression. Vehicle-transplanted hearts were used as a negative control for GFP expression. GFP expression levels in left ventricle (LV) of transplanted animals were determined at 1, 7 and 21 days post-RCV. A time-dependent decline in GFP expression level in LV was observed (see FIG. 3C). At one day post-RCV infusion, the highest level of GFP expression was observed in the LV (see FIG. 3D), compared to the rest of myocardial chambers and the scar area. At 7 days and 21 days, the highest GFP expression level was detected in LV (FIG. 3D). These data indicated that RCV-delivered cells were retained in CHF heart. The exogenous cells were identified in the myocardium by GFP fluorescence (FIGS. 3B and 3E). GFP+ cells were found mainly in the scar border zone and remote LV area (FIG. 3E); however, a small number of cells were detected in the RV. GFP was co-localized with SMA and with αMHC suggesting that transplanted c-Kit+ cells contributed to neovascularization and cardiomyogenesis (FIGS. 4A and 4B). Finally, some GFP+ cells maintained expression of c-Kit indicating the presence of undifferentiated exogenous c-Kit+ cells (see FIG. 4C). These results showed that the methods of the present invention are useful for delivering stem cells to the heart. These results further showed that the methods of the present invention are useful for treating heart disease.

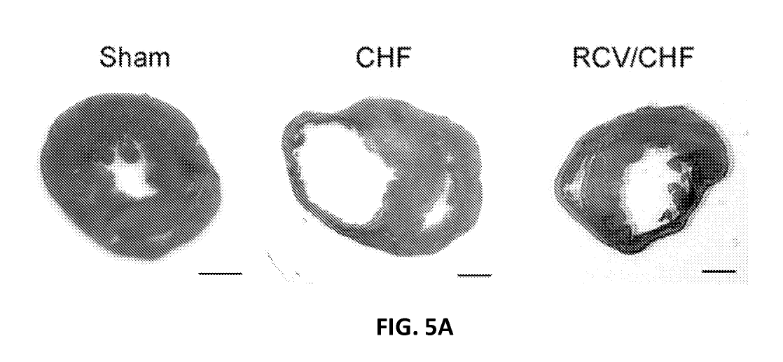

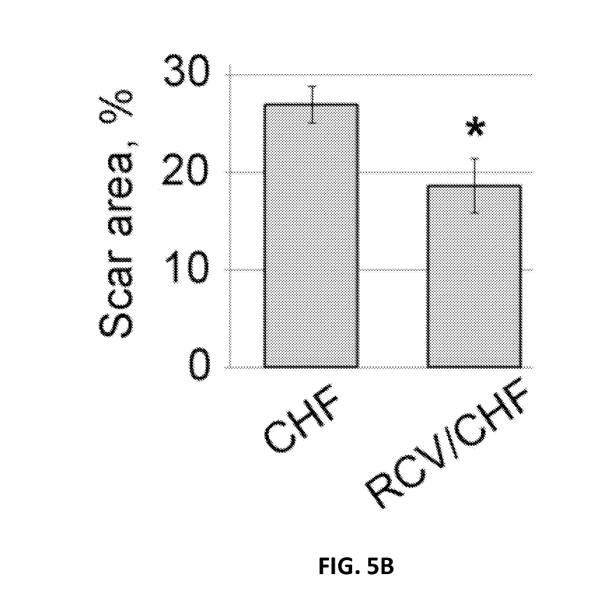

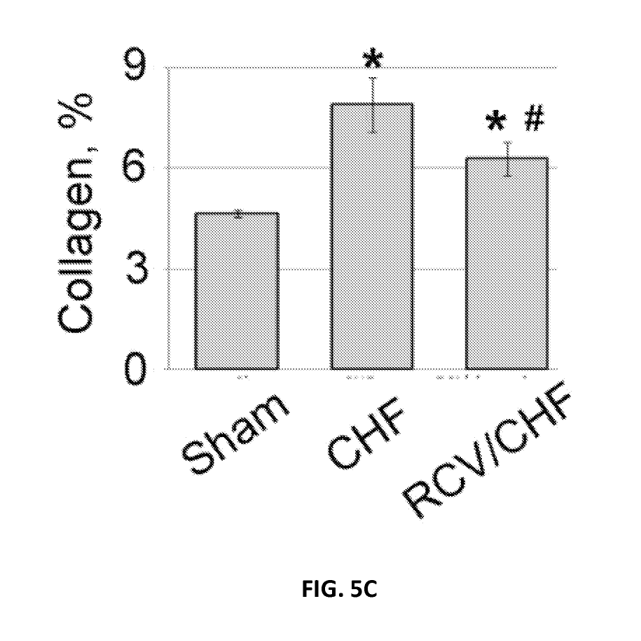

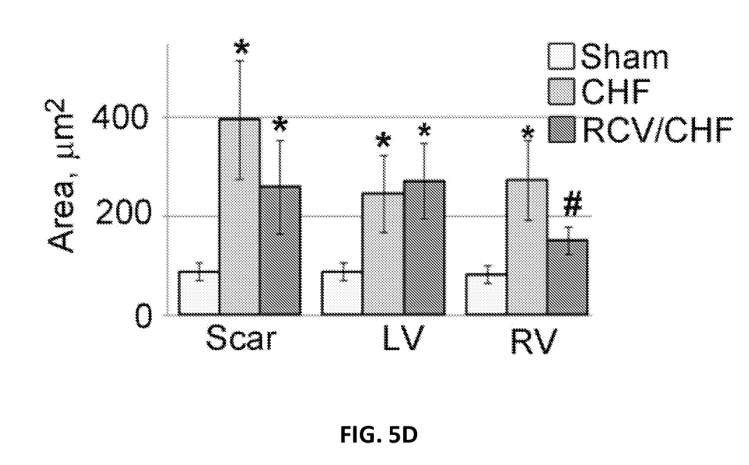

The effects of RCV transplanted c-Kit+ cells on heart remodeling were also evaluated. At 21-days post RCV infusion, scar size was significantly smaller in CHF animals treated with c-Kit+ cells compared to vehicle-treated CHF controls (see FIGS. 5A and 5B). After 21 days, cell treatment resulted in a significant decrease in total collagen amount when compared to vehicle-treated CHF controls (FIG. 5C). The effect of transplanted c-Kit+ cells on angiogenesis in CHF hearts was assessed. A significantly higher number of vWf+ capillaries were found in the infarct zone of cell-transplanted animals compared to vehicle-treated CHF controls (FIG. 5E). In cell-treated CHF rats, the average cardiomyocyte's cross-section area was found to be smaller in both the scar border zone and right ventricle compared to vehicle-treated CHF animals, suggesting that stem cell treatment resulted in a decrease in cardiomyocyte hypertrophy (see FIG. 5D). Together these data indicate that RCV-infused c-Kit+ cells retarded CHF heart remodeling. These results showed that the methods of the present invention are useful for delivering stem cells to the heart. These results further showed that the methods of the present invention are useful for treating heart disease.

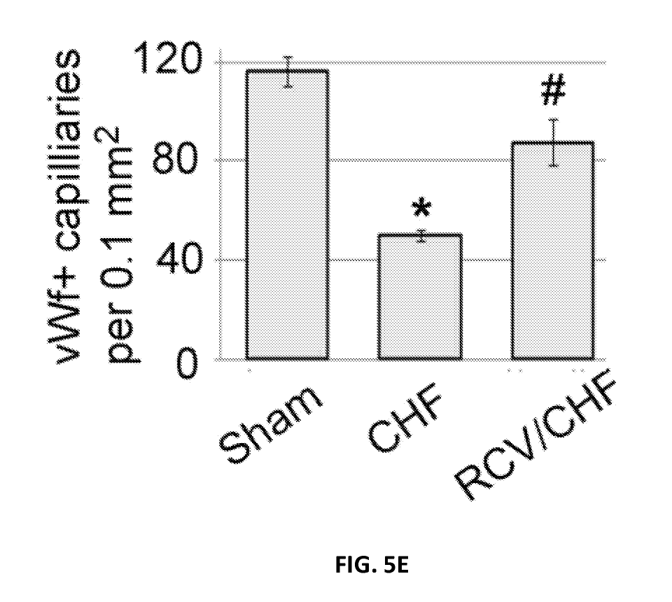

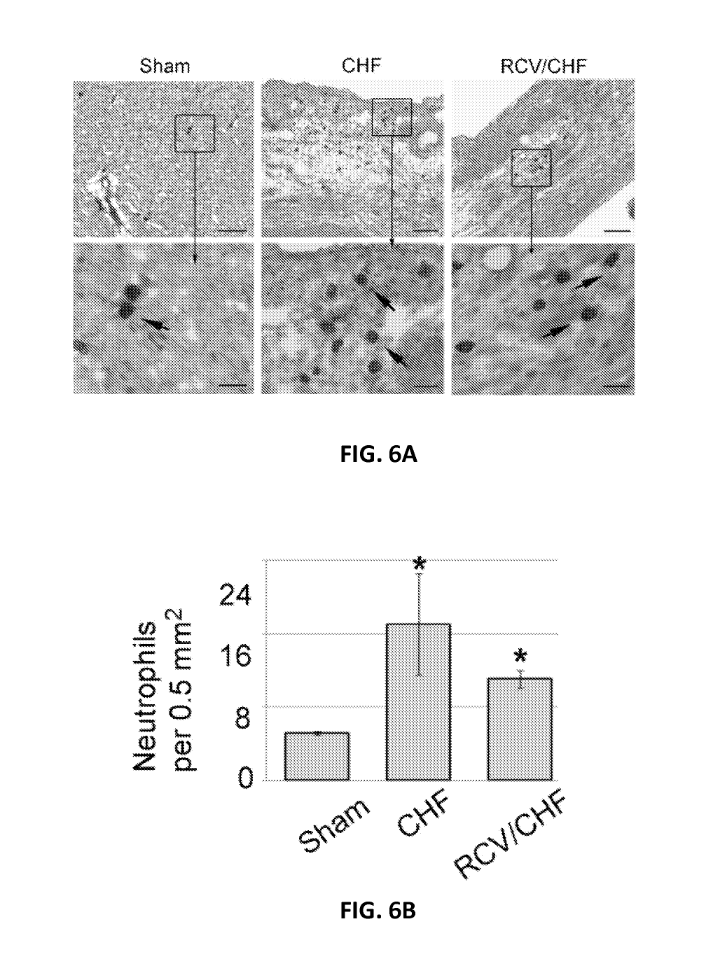

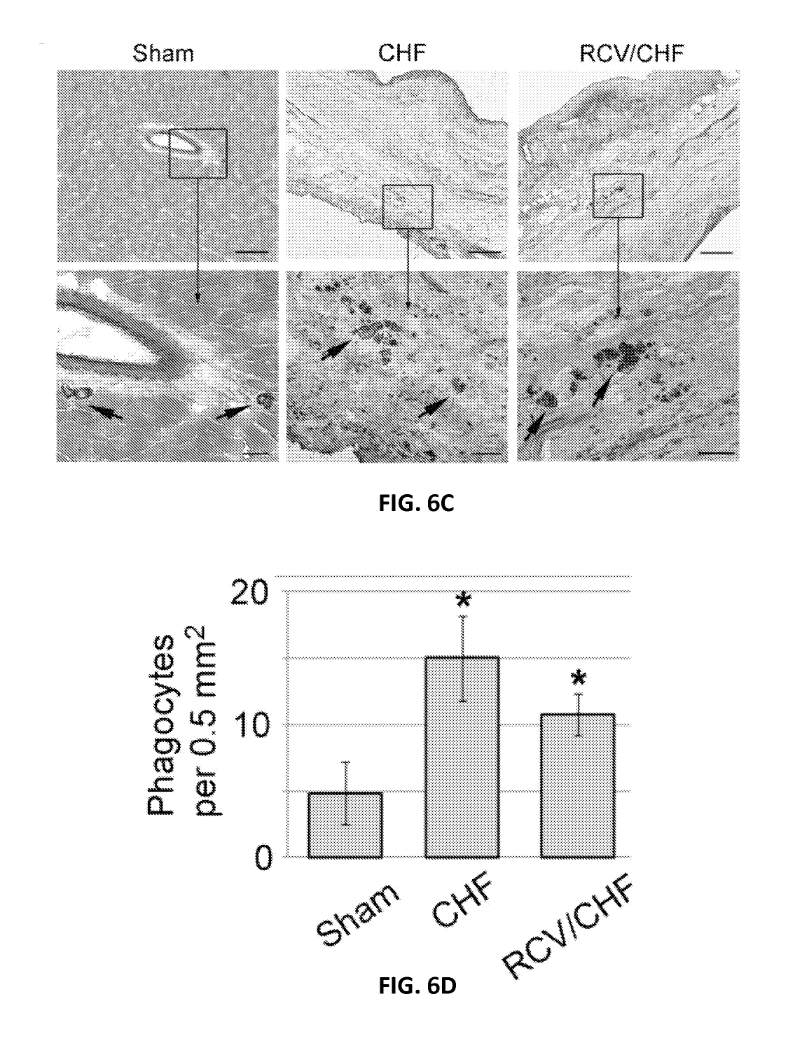

Host immune reaction against transplanted c-Kit+ cells was examined at 21 days after RCV infusion. The number of CD68+ phagocytes and neutrophils was quantified in the infarct zone of both vehicle- and cell-treated CHF rats and in LV of vehicle- and cell-treated sham rats (see FIGS. 6A and 6C). There was no statistical difference in the number of both neutrophil and CD68+ phagocytes between vehicle- and cell-treated sham-operated animals. Compared to both sham groups, the number of neutrophils or CD68+ phagocytes was significantly higher in both vehicle and cell-treated CHF animals (FIGS. 6B and 6D). Additionally, there was no statistical differences in the number of CD68+ cells or neutrophils between cell- and vehicle-treated CHF animals; however, the average number of neutrophils tended to be lower in RCV-transplanted rats compared to CHF controls (FIGS. 6B and 6D). A similar number of immune cells in infarcted tissues of vehicle- and cell-treated CHF animals suggested that the observed host immune reaction was mainly due to MI-induced ischemic injury rather than cell transplantation. In addition, these data suggest that at 21 days post transplantation, RCV-delivered c-Kit+ cells were not rejected by both sham-operated or CHF hosts. These results showed that the methods of the present invention are useful for delivering stem cells to the heart. These results further showed that the methods of the present invention are useful for treating heart disease.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A method of retrograde coronary vein delivery of stem cells to a subject's heart comprising the steps of: inserting a catheter into a right atrium of the heart; occluding one or more blood vessels of the heart; and infusing through the catheter a solution comprising stem cells to the atrium of the subject's heart, thereby delivering stem cells to the subject's heart.

2. The method of claim 1, wherein the one or more blood vessels is selected from the group consisting of inferior vena cava, superior vena cava, and pulmonary artery.

3. The method of claim 1, wherein the solution is infused for approximately 5 seconds, approximately 10 seconds, approximately 20 seconds, approximately 30 seconds, approximately 40 seconds, approximately 50 seconds, approximate 1 minute, approximately 2 minutes, approximately 5 minutes, approximately 10 minutes, approximately 20 minutes, or approximately 30 minutes.

4. The method of claim 1, wherein the volume of the solution is approximately 100 ul, 200 ul, 300 ul, 400 ul, 500 ul, 1 ml, 2 ml, 3 ml, 4 ml, 5 ml, 10 ml, 20 ml, 30 ml, 40 ml, or 50 ml.

5. The method of claim 1, wherein the number of stem cells in the solution comprises approximately 100 thousand, 250 thousand, 500 thousand, 1 million, 2 million, 3 million, 4 million, 5 million, 10 million, 20 million, or 50 million.

6. The method of claim 1, wherein the stem cells express c-Kit.

7. The method of claim 6, wherein the stem cells do not express CD45 or CD34.

8. The method of claim 6, wherein the stem cells further express Nanog, Flk-1/KDR, and Ki67.

9. The method of claim 6, wherein the stem cells further express one or more markers comprising Nanog, Sox1, Oct3/4, Isl1, Nkx2.5, GATA4.

10. The method of claim 1, wherein the solution further comprises serum-free DMEM medium.

11. The method of claim 1 further comprising, treating the stem cells with one or more of the agents selected from the group consisting of TGF-beta, mocetinostat, and VEGF.

12. The method of claim 1, wherein the stem cells are progenitor cells isolated from cardiac explant-derived cells.

13. A method for treating a subject having or suspected of having a heart disease, the method comprising, inserting a catheter into a right atrium of the heart; occluding one or more blood vessels of the heart; and infusing through the catheter a solution comprising stem cells to the atrium of the subject's heart, thereby treating the subject.

14. The method of claim 12, wherein the subject has a heart disease selected from the group consisting of chronic heart failure, myocardial infarction, congestive heart failure, congenital heart disease, cardiomyopathy, pericarditis, angina, and coronary artery disease.
